SEQUENCE LISTING (15 sequences)

SEQ ID NO: 1 - protein, 117 aa, synthetic construct
QVQLQESGPGLVKPSETLSLTCTVSGYSISSGYGWSWIRQPPGKGLEWIGYISYYGSISYNPSLKSRVTISVDTSKNQFSLKLSSVTAADTAVYYCARTNLLYFDSWGQGTLVTVSS

SEQ ID NO: 2 - protein, 108 aa, synthetic construct
EIVLTQSPATLSLSPGERATLSCRASQSVSNNLAWYQQKPGQAPRLLIYDASSRATGIPARFSGSGSGTDFTLTISSLEPEDFAVYYCQQYSDWPQYTFGQGTKVEIK

SEQ ID NO: 3 - protein, 120 aa, synthetic construct
QVQLVQSGAEVKKPGSSVKVSCKASGFTFGSYGINWVRQAPGQGLEWMGVIVPLVGVTIYAEKFQGRVTITADESTSTAYMELSSLRSEDTAVYYCARGQEYFYWYFDLWGQGTLVTVSS

SEQ ID NO: 4 - protein, 111 aa, synthetic construct
QSALTQPPSASGSPGQSVTISCSGSSSDIGGYNRVSWYQQHPGKAPKLMIYDVSKRPSGVPDRFSGSKSGNTASLTVSGLQAEDEADYYCSSYAGTDTFILFGGGTKLTVL

SEQ ID NO: 5 - protein, 118 aa, synthetic construct
QVQLQESGPGLVKPSETLSLTCTVTGYSFTSGYSWHWIRQPPGKGLEWIGYIHDSGGTNYNPSLKSRVTISRDTSKNQFSLKLSSVTAADTAVYYCAREENYRYFDVWGQGTLVTVSS

SEQ ID NO: 6 - protein, 106 aa, synthetic construct
EIVLTQSPATLSLSPGERATLSCSASSSVSYMHWFQQKPGQSPRLLIYSTSNLASGIPARFSGSGSGTDFTLTISSLEPEDFAVYYCQQRSSYPPTFGQGTKVEIK

SEQ ID NO: 7 - protein, 118 aa, synthetic construct
QVQLQESGPGLVKPSETLSLTCAVSGYSFTSGYSWHWIRQPPGKGLEWIGYIHDSGGTNYNPSLKSRVTISRDTSKNQFSLKLSSVTAADTAVYYCAREENYRYFDVWGQGTLVTVSS

SEQ ID NO: 8 - protein, 106 aa, synthetic construct; identical to SEQ ID NO: 6

SEQ ID NO: 9 - protein, 118 aa, synthetic construct
EVQLVESGGGLVKPGGSLRLSCAASGFTFSTYSMNWVRQAPGKGLEWVSYISHSSDKIYYADSVKGRFTISRDNAKNSLYLQMNSLRDEDTAVYYCVRSWGVVGFDLWGRGTLVTVSS

SEQ ID NO: 10 - protein, 111 aa, synthetic construct
EIVLTQSPLTLPVTPGEPASISCRSSQSLLHSSGYNYLDWYLQKPGQSPQLLIYLGSNRASGVPDRFSGSGSGTDFTLKISRVEAEDVGVYYCMQALQVPTFGPGTKVDIK

SEQ ID NO: 11 - protein, 118 aa, synthetic construct
EVQLVESGGGLVKPGGSLRLSCAASGFTFSTYSMNWVRQAPGKGLEWVSYISHSSDIIYYADSVKGRFTISRDNAKNSLYLQMNSLRDEDTAVYYCVRSWGKVGFDLWGRGPLVTVSS

SEQ ID NO: 12 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 107 aa, synthetic construct [sequence omitted]

(19) United States
(12) Patent Application Publication
(10) Pub. No.: US 2026/0308245 A1
(43) Pub. Date: Oct. 8, 2026

(54) A HIGH-CONCENTRATION MONOCLONAL ANTIBODY FORMULATION AND USES THEREOF

(71) Applicant: Jiangsu BioJeTay Biotechnology Co., Ltd., Jiangsu (CN)

(72) Inventors: Qinglei LI, Daxing District, Beijing (CN); Chuan LIAO, Daxing District, Beijing (CN)

(73) Assignee: Jiangsu BioJeTay Biotechnology Co., Ltd., Jiangsu (CN)

(21) Appl. No.: 18/996,786

(22) PCT Filed: Jul. 27, 2023
(86) PCT No.: PCT/CN2023/109456

§ 371 Date: Jan. 17, 2025

(30) Foreign Application Priority Data
Jul. 29, 2022 (CN) 202210902908.6

(51) Int. Cl. C07K 16/24; A61K 39/00; A61K 47/10; A61K 47/14; A61K 47/22; A61K 47/26; C07K 16/28

(52) U.S. Cl. CPC C07K 16/244; A61K 47/10; A61K 47/14; A61K 47/22; A61K 47/26; C07K 16/283; A61K 2039/545; C07K 2317/56; C07K 2317/622

(57) ABSTRACT

Provided is a high-concentration antibody formulation. The antibody formulation includes a monoclonal antibody and surfactants. The surfactants are polysorbate and poloxamer. The concentration of the monoclonal antibody is 100 mg/mL to 200 mg/mL. The high-concentration antibody formulation is high in stability under freeze thawing, oscillation, acceleration and long-term conditions, such that the high-concentration antibody formulation can keep good stability in the preparation, transportation and storage processes, thereby guaranteeing the clinical medication safety and quality controllability.

### FIELD OF THE APPLICATION

The present invention relates to the field of biomedicine technology, and is especially a high-concentration monoclonal antibody formulation and uses thereof.

### BACKGROUND OF THE APPLICATION

Antibodies, as protein drugs, compared to conventional chemical drugs, are affected by many environmental factors during storage, such as high sensitivity to temperature, oxygen, and ultraviolet light. Because of these factors, a variety of physical or chemical changes may occur. Physical changes include: degradation, aggregation (soluble and insoluble polymers); chemical changes include: hydrolysis, oxidation and so on. These changes can lead to the inability of the antibodies to ensure their stability dure storage, resulting in the following effects: on the one hand, changes in actual dosage can affect efficacy, and on the other hand, the production of related substances can cause side effects.

Especially, the stability of high-concentration antibody formulation is more difficult to maintain. Nonionic surfactants are widely used in the production, transportation, freezing and thawing and long-term storage of biologicals to prevent protein aggregation and particle formation. Polysorbate 20 and Polysorbate 80 are the most commonly used nonionic surfactants in biologicals. However, the chemical properties of Polysorbate 20 and 80 (PS20, PS80) were found to be unstable and often oxidized and hydrolyzed. Hydrolysis of polysorbate can form visible or invisible particles in the formulation, making the pharmaceutical formulations unsuitable for injection; oxidation of polysorbate can lead to a decrease in concentration that does not achieve the expected effect, and can also produce harmful substances such as peroxides, aldehydes, and organic acids, which can cause changes in proteins. Poloxamer 188 (P188) is the only surfactant used for injectable biologics on the market, apart from polysorbate. In addition to compete with protein molecules in the formulation to bind to the hydrophobic layer, it can also directly bind to protein molecules to prevent protein aggregation and precipitation.

At present, there are more than 100 kinds of antibody drugs on the market, of which there are dozens of high-concentration antibody (>140 mg/mL) drugs, but all of them use a single surfactant. Through research, the applicant of the present invention has found that due to the special properties of the high concentration antibody, a large number of visible particles can be formed in a very short period of time under the condition of oscillation when only the surfactant polysorbate is used. Although poloxamer can effectively control the formation of aggregation in a certain time, it can not protect the stability of antibodies in many aspects. The present invention uses dual surfactants (polysorbate and poloxamer) to protect the target antibody. Compared with a single surfactant, the formulation containing dual surfactants can maintain good stability of the high concentration antibody.

### DESCRIPTION OF THE APPLICATION

The present application provides a high concentration antibody formulation, and the antibody formulation comprises a monoclonal antibody and surfactants, wherein the surfactants are polysorbate and poloxamer, and the concentration of the monoclonal antibody is from 100 mg/ml to 200 mg/ml, optionally from 135 mg/ml to 165 mg/ml. In some specific embodiments, the concentration of the monoclonal antibody is 100 mg/ml, 120 mg/ml, 135 mg/ml, 140 mg/ml, 145 mg/ml, 150 mg/ml, 155 mg/ml, 160 mg/ml, 165 mg/ml, 180 mg/ml, 190 mg/ml or 200 mg/ml.

In some embodiments of the present application, the polysorbate is polysorbate 20 and/or polysorbate 80; optionally, the poloxamer is poloxamer 188.

In some embodiments of the present application, the concentration of the polysorbate is from 0.02 mg/ml to 0.45 mg/ml, optionally from 0.08 mg/ml to 0.4 mg/ml, more optionally from 0.1 mg/ml to 0.3 mg/ml. In some specific embodiments of the present application, the concentration of the polysorbate is 0.02 mg/ml, 0.03 mg/ml, 0.05 mg/ml, 0.06 mg/ml, 0.08 mg/ml, 0.10 mg/ml, 0.12 mg/ml, 0.15 mg/ml, 0.2 mg/ml, 0.25 mg/ml, 0.3 mg/ml, 0.35 mg/ml, 0.4 mg/ml or 0.45 mg/ml.

In some embodiments of the present application, the concentration of the poloxamer is from 0.4 to 1.5 mg/ml, optionally from 0.8 mg/ml to 1.2 mg/ml, more optionally 1.0 mg/ml; in some specific embodiments of the present application, the concentration of the poloxamer is 0.4 mg/ml, 0.5 mg/ml, 0.6 mg/ml, 0.8 mg/ml, 1.0 mg/ml, 1.2 mg/m or 1.5 mg/ml.

In some embodiments of the present application, the antibody formulation further comprises a stabilizer, optionally, the stabilizer is any one or more of sucrose, trehalose, maltose, sorbitol, mannitol, sodium chloride, arginine, glycine, proline and lysine; more optionally, the stabilizer is any one or more of sucrose, trehalose, sodium chloride or arginine.

In some embodiments of the present application, the stabilizer is sucrose, trehalose, maltose, sorbitol and/or mannitol at a concentration of from 40 mg/ml to 80 mg/ml, optionally from 50 mg/ml to 80 mg/ml, and further optionally from 50 mg/ml to 70 mg/ml, or sodium chloride, arginine, glycine, proline or lysine at a concentration of from 100 mM to 180 mM, preferably from 130 mM to 150 mM; in some specific embodiments of the present application, the stabilizer is sucrose, trehalose, maltose, sorbitol and/or mannitol at a concentration of 40 mg/ml, 50 mg/ml, 60 mg/ml, 70 mg/ml or 80 mg/ml, or the stabilizer is sodium chloride, arginine, glycine, proline or lysine at a concentration 100 mM, 110 mM, 120 mM, 130 mM, 140 mM, 150 mM, 160 mM, 170 mM or 180 mM.

In some embodiments of the present application, the antibody formulation further comprises a buffer, optionally the buffer is citrate buffer, acetate buffer and/or histamine buffer.

In some embodiments of the present application, the concentration of the buffer is from 3 mM to 50 mM; optionally, the concentration of the buffer is from 10 mM to 30 mM; in some specific embodiments of the present application, the concentration of the buffer is 3 mM, 5 mM, 8 mM, 10 mM, 15 mM, 20 mM, 25 mM or 30 mM.

In some embodiments of the present application, the pH of the formulation is from 4.8 to 5.5, optionally from 5.0 to 5.4; in some specific embodiments of the present application, the pH of the formulation is 4.8, 4.9, 5.0, 5.1, 5.2, 5.3, 5.4 or 5.5.

In some embodiments of the present application, the concentration of the antibody is from 100 mg/ml to 200 mg/ml; the stabilizer is sucrose and/or trehalose of from 40 mg/ml to 80 mg/ml, or the stabilizer is sodium chloride or arginine of from 100 mM to 180 mM; the buffer is acetate buffer, citrate buffer and/or histidine buffer of from 3 mM to 50 mM; the pH value of the formulation is 4.8-5.5; the surfactants are polysorbate of from 0.02 mg/ml to 0.45 mg/ml and poloxamer of from 0.4 mg/ml to 1.5 mg/ml.

In some embodiments of the present application, the concentration of the antibody is from 135 mg/ml to 165 mg/ml; the stabilizer is sucrose and/or trehalose of from 50 mg/ml to 80 mg/ml, or the stabilizer is sodium chloride or arginine of from 130 mM to 150 mM; the buffer is acetate buffer, citrate buffer and/or histidine buffer of from 10 mM to 30 mM; the pH value of the formulation is from 4.8 to 5.5; the surfactants are polysorbate of from 0.02 mg/ml to 0.45 mg/ml and poloxamer of from 0.4 mg/ml to 1.5 mg/ml.

In some embodiments of the present application, the concentration of the antibody is from 135 mg/ml to 165 mg/ml; the stabilizer is sucrose and/or trehalose of from 50 mg/ml to 70 mg/ml, or the stabilizer is sodium chloride or arginine of from 130 mM to 150 mM; the buffer is acetate buffer, citrate buffer and/or histidine buffer of from 10 mM to 30 mM; the pH value of the formulation is from 5.0 to 5.4; the surfactants are polysorbate of from 0.02 mg/ml to 0.45 mg/ml and poloxamer of from 0.4 mg/ml to 1.5 mg/ml.

In some embodiments of the present application, the concentration of the antibody is from 135 mg/ml to 165 mg/ml; the stabilizer is sucrose of from 50 mg/ml to 70 mg/ml; the buffer is acetate buffer of from 10 mM to 30 mM; the pH value of the formulation is from 5.0 to 5.4; the surfactants are polysorbate of from 0.02 mg/ml to 0.45 mg/ml and poloxamer of from 0.4 mg/ml to 1.5 mg/ml.

In some embodiments of the present application, the formulation is any one of the following antibody formulation s:
(1) the concentration of the antibody is 135 mg/ml, 140 mg/ml, 145 mg/ml, 150 mg/ml, 155 mg/ml, 160 mg/ml or 165 mg/ml; the stabilizer is sucrose and/or trehalose of from 50 mg/ml to 70 mg/ml, or the stabilizer is sodium chloride or arginine of from 130 mM to 150 mM; the buffer is acetate buffer, citrate buffer and/or histidine buffer of from 10 mM to 30 mM; the pH value of the formulation is from 5.0 to 5.4; the surfactants are polysorbate of from 0.02 mg/ml to 0.45 mg/ml and poloxamer of from 0.4 mg/ml to 1.5 mg/ml;
(2) the concentration of the antibody is from 135 mg/ml to 165 mg/ml; the stabilizer is sucrose and/or trehalose of 50 mg/ml, 60 mg/ml or 70 mg/ml, or the stabilizer is sodium chloride or arginine of 130 mM, 140 mM or 150 mM; the buffer is acetate buffer, citrate buffer and/or histidine buffer of from 10 mM to 30 mM; the pH value of the formulation is from 5.0 to 5.4; the surfactants are polysorbate of from 0.02 mg/ml to 0.45 mg/ml and poloxamer of from 0.4 mg/ml to 1.5 mg/ml;
(3) the concentration of the antibody is from 135 mg/ml to 165 mg/ml; the stabilizer is sucrose and/or trehalose of from 50 mg/ml to 70 mg/ml, or the stabilizer is sodium chloride or arginine of from 130 mM to 150 mM; the buffer is acetate buffer, citrate buffer and/or histidine buffer of 10 mM, 20 mM or 30 mM; the pH value of the formulation is from 5.0 to 5.4; the surfactants are polysorbate of from 0.02 mg/ml to 0.45 mg/ml and poloxamer of from 0.4 mg/ml to 1.5 mg/ml;
(4) the concentration of the antibody is from 135 mg/ml to 165 mg/ml; the stabilizer is sucrose and/or trehalose of from 50 mg/ml to 70 mg/ml, or the stabilizer is sodium chloride or arginine of from 130 mM to 150 mM; the buffer is acetate buffer, citrate buffer and/or histidine buffer of from 10 mM to 30 mM; the pH value of the formulation is 5.0, 5.1, 5.2, 5.3 or 5.4; the surfactants are polysorbate of from 0.02 mg/ml to 0.45 mg/ml and poloxamer of from 0.4 mg/ml to 1.5 mg/ml;
(5) the concentration of the antibody is from 135 mg/ml to 165 mg/ml; the stabilizer is sucrose and/or trehalose of from 50 mg/ml to 70 mg/ml, or the stabilizer is sodium chloride or arginine of from 130 mM to 150 mM; the buffer is acetate buffer, citrate buffer and/or histidine buffer of from 10 mM to 30 mM; the pH value of the formulation is from 5.0 to 5.4; the surfactants are polysorbate of 0.02 mg/ml, 0.03 mg/ml, 0.04 mg/ml, 0.05 mg/ml, 0.06 mg/ml, 0.08 mg/ml, 0.10 mg/ml, 0.12 mg/ml, 0.15 mg/ml, 0.20 mg/ml, 0.30 mg/ml, 0.40 mg/ml or 0.45 mg/ml and poloxamer of from 0.4 mg/ml to 1.5 mg/ml;
(6) the concentration of the antibody is from 135 mg/ml to 165 mg/ml; the stabilizer is sucrose and/or trehalose of from 50 mg/ml to 70 mg/ml, or the stabilizer is sodium chloride or arginine of from 130 mM to 150 mM; the buffer is acetate buffer, citrate buffer and/or histidine buffer of from 10 mM to 30 mM; the pH value of the formulation is from 5.0 to 5.4; the surfactants are polysorbate of from 0.02 mg/ml to 0.45 mg/ml and poloxamer of 0.4 mg/ml, 0.5 mg/ml, 0.6 mg/ml, 0.8 mg/ml, 1.0 mg/ml or 1.2 mg/ml 1.5 mg/ml;
the polysorbate is polysorbate 80 or polysorbate 20.

In some embodiments of the present application, the formulation is any one of the following antibody formulation s:
(1) the concentration of the antibody is 150 mg/ml; the stabilizer is sodium chloride of 130 mM; the buffer is acetate buffer of 20 mM; the pH value of the formulation is 5.4; the surfactants are polysorbate of 0.1 mg/ml or 0.3 mg/ml and poloxamer of 1.0 mg/ml;
(2) the concentration of the antibody is 150 mg/ml; the stabilizer is sucrose of 60 mg/ml; the buffer is citrate buffer of 20 mM; the pH value of the formulation is 5.0; the surfactants are polysorbate of 0.1 mg/ml or 0.3 mg/ml and poloxamer of 1.0 mg/ml;
(3) the concentration of the antibody is 135 mg/ml; the stabilizer is sucrose of 50 mg/ml; the buffer is acetate buffer of 10 mM; the pH value of the formulation is 5.4; the surfactants are polysorbate of 0.1 mg/ml or 0.3 mg/ml and poloxamer of 1.0 mg/ml;
(4) the concentration of the antibody is 150 mg/ml; the stabilizer is sucrose of 60 mg/ml; the buffer is acetate buffer of 20 mM; the pH value of the formulation is 5.2; the surfactants are polysorbate of 0.1 mg/ml or 0.3 mg/ml and poloxamer of 1.0 mg/ml;
(5) the concentration of the antibody is 165 mg/ml; the stabilizer is the sucrose of 70 mg/ml; the buffer is the acetate buffer of 30 mM; the pH value of the formulation is 5.0; the surfactants are polysorbate of 0.1 mg/ml or 0.3 mg/ml and poloxamer of 1.0 mg/ml;
(6) the concentration of the antibody is 150 mg/ml; the stabilizer is the sucrose of 80 mg/ml; the buffer is the acetate buffer of 30 mM; the pH value of the formulation is 5.4; the surfactants are polysorbate of 0.1 mg/ml or 0.3 mg/ml and poloxamer of 1.0 mg/ml;
(7) the concentration of the antibody is 150 mg/ml; the stabilizer is arginine of 150 mM; the buffer is acetate buffer of 20 mM; the pH value of the formulation is 5.2; the surfactants are polysorbate of 0.1 mg/ml or 0.3 mg/ml and poloxamer of 1.0 mg/ml;
(8) the concentration of the antibody is 160 mg/ml; the stabilizer is sucrose of 60 mg/ml; the buffer is citrate buffer of 20 mM; the pH value of the formulation is 5.0; the surfactants are polysorbate of 0.1 mg/ml or 0.3 mg/ml and poloxamer of 1.0 mg/ml;
(9) the concentration of the antibody is 150 mg/ml; the stabilizer is the sucrose of 60 mg/ml; the buffer is the citrate buffer of 20 mM; the pH value of the formulation is 4.8; the surfactants are polysorbate of 0.1 mg/ml or 0.3 mg/ml and poloxamer of 1.0 mg/ml;
(10) the concentration of the antibody is 150 mg/ml; the stabilizer is the sucrose of 60 mg/ml; the buffer is the histamine salt buffer of 20 mM; the pH value of the formulation is 5.5; the surfactants are polysorbate of 0.1 mg/ml or 0.3 mg/ml and poloxamer of 1.0 mg/ml;
(11) the concentration of the antibody is 150 mg/ml; the stabilizer is sucrose of 60 mg/ml; the buffer is histamine salt buffer of 20 mM; the pH value of the formulation is 5.2; the surfactants are polysorbate of 0.02 mg/ml or 0.05 mg/ml and poloxamer of 1.5 mg/ml;
(12) the concentration of the antibody is 150 mg/ml; the stabilizer is the sucrose of 60 mg/ml; the buffer is the histidine salt buffer of 20 mM; the pH value of the formulation is 5.2; the surfactants are polysorbate of 0.15 mg/ml and poloxamer of 0.5 mg/ml;
(13) the concentration of the antibody is 150 mg/ml; the stabilizer is the sucrose of 60 mg/ml; the buffer is the histidine salt buffer of 20 mM; the pH value of the formulation is 5.2; the surfactants are polysorbate of 0.45 mg/ml and poloxamer of 0.4 mg/ml;
the polysorbate is polysorbate 80 or polysorbate 20.

In some embodiments of the present application, the antibody formulation further comprises an antioxidant and/or a preservative.

The preservative or antioxidant is a preservative or an antioxidant commonly used in antibody formulations. The concentration of the antioxidant and/or preservative is from 0 to 20 mM, and in some specific embodiments, the concentration of the antioxidant is 0, 5 mM, 8 mM, 10 mM, 12 mM, 15 mM, 18 mM or 20 mM. In some embodiments, the preservative is ethylenediamine tetraacetic acid (EDTA), diethylenetriamine pentaacetic acid (DTPA) or a combination thereof; the antioxidant is methionine.

In some embodiments of the present application, the antibody formulation is a liquid formulation or a powder injection for injection.

In some embodiments of the present application, the antibody is an antibody that specifically recognizes thymic stromal lymphopoietin (TSLP) or an antibody that specifically recognizes the neonatal Fc receptor (FcRn). On the other hand, the present invention also provides the use of the antibody formulation of above in the preparation of medicament for the treatment of diseases; optionally, the diseases include inflammatory or autoimmune diseases.

On the other hand, the antibody formulation provided by the present application is suitable for a variety of modes of administration known in the art, including but not limited to: intravenous, intra-arterial, intraperitoneal, intrapulmonary, intravascular, intramuscular, endotracheal, subcutaneous, intraocular, intrathecal, mucosal or percutaneous. In some embodiments, the formulation is administered subcutaneously. In some embodiments, the formulation is administered through the muscle.

The “antibody” of the present application includes full-length antibodies and antigen-binding fragments thereof. A full-length antibody comprises two heavy chains and two light chains. The variable regions of the light and heavy chains are responsible for antigen binding. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains. Respectively, several of the major antibody classes are divided into subclasses such as IgG1 (γ1 heavy chain), IgG2 (γ2 heavy chain), IgG3 (γ3 heavy chain), IgG4 (γ4 heavy chain), IgA1 (α1 heavy chain), or IgA2 (α2 heavy chain).

The term “antigen-binding fragment” as used herein includes an antibody fragment including, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain Fv (scFv), an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure.

The high-concentration antibody formulation provided by the present application has the following excellent effects:

The antibody preparation provided by the present application adopts the combination of two surfactants of polysorbate and poloxamer, which can more effectively prevent the collision contact between antibody molecules and reduce the formation of polymers at the interface compared with the use of a single surfactant. The high-concentration antibody formulation of the present application can maintain strong stability under freeze-thaw, oscillation, acceleration and long-term conditions, and can maintain good stability during preparation, transportation and storage, ensuring the safety of clinical drug use and quality control.

### EXAMPLES

In order to make the technical problems to be solved by the present application, the technical scheme adopted and the advantages more clear, the present invention will be described in detail in combination with specific embodiments. The following examples are used to illustrate the invention, but not to limit the scope of the invention.

The reagents used in the following examples are prepared by conventional methods or obtained commercially unless otherwise specified; the experimental methods used are conventional methods, unless otherwise specified; the materials, instruments, etc. used are obtained by commercial means unless otherwise specified.

The exemplary antibodies used in the examples of the present i application are respectively TSLP-01-IgG1, TSLP-02-IgG1, FcRn-01-IgG4, FcRn-02-IgG4, FcRn-03-IgG4 and FcRn-04-IgG4, and their amino acid sequences are shown in Table 1:

TABLE 1
Amino acid sequences of antibody

| | Sequences |
|---|---|
| TSLP-01-IgG1 | QVQLQESGPGLVKPSETLSLTCTVSGYSISSGYGWSWIRQP |
| V_{H} | PGKGLEWIGYISYYGSISYNPSLKSRVTISVDTSKNQFSLKL |
| | SSVTAADTAVYYCARTNLLYFDSWGQGTLVTVSS |
| | (SEQ ID NO: 1) |
| TSLP-01-IgG1 | EIVLTQSPATLSLSPGERATLSCRASQSVSNNLAWYQQKPG |
| V_{L} | QAPRLLIYDASSRATGIPARFSGSGSGTDFTLTISSLEPEDFA |
| | VYYCQQYSDWPQYTFGQGTKVEIK |
| | (SEQ ID NO: 2) |
| TSLP-02-IgG1 | QVQLVQSGAEVKKPGSSVKVSCKASGFTFGSYGINWVRQ |
| V_{H} | APGQGLEWMGVIVPLVGVTIYAEKFQGRVTITADESTSTA |
| | YMELSSLRSEDTAVYYCARGQEYFYWYFDLWGQGTLVT |
| | VSS |
| | (SEQ ID NO: 3) |
| TSLP-02-IgG1 | QSALTQPPSASGSPGQSVTISCSGSSSDIGGYNRVSWYQQH |
| V_{L} | PGKAPKLMIYDVSKRPSGVPDRESGSKSGNTASLTVSGLQ |
| | AEDEADYYCSSYAGTDTFILFGGGTKLTVL |
| | (SEQ ID NO: 4) |
| FcRn-01-IgG4 | QVQLQESGPGLVKPSETLSLTCTVTGYSFTSGYSWHWIRQ |
| V_{H} | PPGKGLEWIGYIHDSGGTNYNPSLKSRVTISRDTSKNQFSL |
| | KLSSVTAADTAVYYCAREENYRYFDVWGQGTLVTVSS |
| | (SEQ ID NO: 5) |
| FcRn-01-IgG4 | EIVLTQSPATLSLSPGERATLSCSASSSVSYMHWFQQKPGQ |
| V_{L} | SPRLLIYSTSNLASGIPARFSGSGSGTDFTLTISSLEPEDFAV |
| | YYCQQRSSYPPTFGQGTKVEIK |
| | (SEQ ID NO: 6) |
| FcRn-02-IgG4 | QVQLQESGPGLVKPSETLSLTCAVSGYSFTSGYSWHWIRQ |
| V_{H} | PPGKGLEWIGYIHDSGGTNYNPSLKSRVTISRDTSKNQFSL |
| | KLSSVTAADTAVYYCAREENYRYFDVWGQGTLVTVSS |
| | (SEQ ID NO: 7) |
| FcRn-02-IgG4 | EIVLTQSPATLSLSPGERATLSCSASSSVSYMHWFQQKPGQ |
| V_{L} | SPRLLIYSTSNLASGIPARFSGSGSGTDFTLTISSLEPEDFAV |
| | YYCQQRSSYPPTFGQGTKVEIK |
| | (SEQ ID NO: 8) |
| FcRn-03-IgG4 | EVOLVESGGGLVKPGGSLRLSCAASGFTFSTYSMNWVRQ |
| V_{H} | APGKGLEWVSYISHSSDKIYYADSVKGRFTISRDNAKNSL |
| | YLQMNSLRDEDTAVYYCVRSWGVVGFDLWGRGTLVTVS |
| | S |
| | (SEQ ID NO: 9) |
| FcRn-03-IgG4 | EIVLTQSPLTLPVTPGEPASISCRSSQSLLHSSGYNYLDWYL |
| V_{L} | QKPGQSPQLLIYLGSNRASGVPDRFSGSGSGTDFTLKISRV |
| FcRn-04-IgG4 | EAEDVGVYYCMQALQVPTFGPGTKVDIK |
| V_{L} | (SEQ ID NO: 10) |
| FcRn-04-IgG4 | EVOLVESGGGLVKPGGSLRLSCAASGFTFSTYSMNWVRQ |
| V_{H} | APGKGLEWVSYISHSSDIIYYADSVKGRFTISRDNAKNSLY |
| | LQMNSLRDEDTAVYYCVRSWGKVGFDLWGRGPLVTVSS |
| | (SEQ ID NO: 11) |
| IgG1 heavy | ASTKGPSVFPLAPSSKSTSGGTAALGCLVKDYFPEPVTVS |
| chain constant | WNSGALTSGVHTFPAVLQSSGLYSLSSVVTVPSSSLGTQTY |
| region | ICNVNHKPSNTKVDKKVEPKSCDKTHTCPPCPAPELLGGP |
| | SVFLFPPKPKDTLMISRTPEVTCVVVDVSHEDPEVKFNWY |
| | VDGVEVHNAKTKPREEQYNSTYRVVSVLTVLHQDWLNG |
| | KEYKCKVSNKALPAPIEKTISKAKGQPREPQVYTLPPSREE |
| | MTKNQVSLTCLVKGFYPSDIAVEWESNGQPENNYKTTPPV |
| | LDSDGSFFLYSKLTVDKSRWQQGNVFSCSVMHEALHNHY |
| | TQKSLSLSPGK |
| | (SEQ ID NO: 12) |
| IgG4 heavy | ASTKGPSVFPLAPCSRSTSESTAALGCLVKDYFPEPVTVSW |
| chain constant | NSGALTSGVHTFPAVLQSSGLYSLSSVVTVPSSSLGTKTYT |
| region | CNVDHKPSNTKVDKRVESKYGPPCPPCPAPEFLGGPSVFL |
| (S228P) | FPPKPKDTLMISRTPEVTCVVVDVSQEDPEVQFNWYVDG |
| | VEVHNAKTKPREEQFNSTYRVVSVLTVLHQDWLNGKEY |
| | KCKVSNKGLPSSIEKTISKAKGQPREPQVYTLPPSQEEMTK |
| | NQVSLTCLVKGFYPSDIAVEWESNGQPENNYKTTPPVLDS |
| | DGSFFLYSRLTVDKSRWQEGNVFSCSVMHEALHNHYTQK |
| | SLSLSLGK |
| | (SEQ ID NO: 13) |
| IgG4 heavy | ASTKGPSVFPLAPCSRSTSESTAALGCLVKDYFPEPVTVSW |
| chain constant | NSGALTSGVHTFPAVLQSSGLYSLSSVVTVPSSSLGTKTYT |
| region | CNVDHKPSNTKVDKRVESKYGPPCPSCPAPEFLGGPSVFL |
| | FPPKPKDTLMISRTPEVTCVVVDVSQEDPEVQFNWYVDG |
| | VEVHNAKTKPREEQFNSTYRVVSVLTVLHQDWLNGKEY |
| | KCKVSNKGLPSSIEKTISKAKGQPREPQVYTLPPSQEEMTK |
| | NQVSLTCLVKGFYPSDIAVEWESNGQPENNYKTTPPVLDS |
| | DGSFFLYSRLTVDKSRWQEGNVFSCSVMHEALHNHYTQK |
| | SLSLSLGK |
| | (SEQ ID NO: 14) |
| Light chain | RTVAAPSVFIFPPSDEQLKSGTASVVCLLNNFYPREAKVQ |
| constant region | WKVDNALQSGNSQESVTEQDSKDSTYSLSSTLTLSKADY |
| (κ) | EKHKVYACEVTHQGLSSPVTKSFNRGEC |
| | (SEQ ID NO: 15) |

### Example 1. Antibody Preparation and Screening

### 1. Preparation and Activity Detection of Anti-TSLP Antibody

### (1) Preparation of Anti-TSLP Antibody

ScFv antibodies against TSLP targets were screened from the scFv antibody yeast display library, and after further optimization, full-length anti-TSLP-01-IGG1 and TSLP-02-IGG1 antibodies were constructed and screened, and their amino acid sequences were shown in Table 1.

### (2) Inhibition of TSLP Induced Stat5 Activation

Evaluate the efficacy of antibodies TSLP-01-IgG1 and TSLP-02-IgG1 shown in Table 1 in inhibiting human TSLP-stimulated signal transduction and transcriptional activator 5 activation in Ba/F3-stable cells, and perform cell analysis. As shown in Table 2, TSLP-01-IgG1 antibodies and TSLP-02-IgG1 antibodies showed better efficacy in inhibiting human TSLP-induced Stat5 activation than the reference antibody AMG157.

### (3) Inhibition of TSLP Induced Ba/F3 Cells Proliferation

Evaluate the ability of anti-TSLP antibodies to inhibit TSLP-induced proliferation of Ba/F3 cells expressing human TSLP receptor (hTSLPR) and human IL-7Ra (hIL7Ra).As shown in Table 2, the anti-TSLP antibodies exhibited better or comparable efficacy in inhibiting TSLP induced Ba/F3 cells proliferation compared to the reference antibody AMG157.

### (4) Inhibition of TARC Release in Human PBMCs Assay

To determine if anti-TSLP antibodies were able to neutralize TSLP in the context of a primary cell driven response, human TSLP induced TARC secretion from human PBMCs was tested in the presence or absence of anti-TSLP antibodies. As shown in Table 2, TSLP-01-IGG1 antibodies and TSLP-02-IGG1 antibodies can inhibite the ability of human TSLP (hTSLP) to promote TARC release from human PBMC, and it also showed improved ability to inhibit human TSLP promoting TARC release as compared to the reference antibody AMG157 (which is described in U.S. Pat. No. 7,982,016).

TABLE 2
Test results of anti-TSLP antibody activity

| | | IC50 of | IC50 of |
|---|---|---|---|
| | IC50 of | inhibition | inhibition |
| | inhibition | Ba/F3 | TARC |
| | Stat5 | cells | release |
| | activation | proliferation | assay |
| Antibodies | (ng/mL) | (ng/mL) | (ng/mL) |
| TSLP-01-IgG1 | 8.844 | 13.61 | 0.7582 |
| TSLP-02-IgG1 | 2.57 | 13.12 | 0.3645 |
| AMG157 | 26.37 | 152.6 | 1.031 |

### 2. Preparation and Activity Detection of Anti-FcRn Antibody

### (1) Preparation of Anti-FcRn Antibody

Balb/c mice were immunized with recombinant human FcRn and Freund adjuvant to establish a murine-derived phage display library of scFv antibody. After screening, identification and optional humanization, full-length antibodies FcRn-01-IgG4 and FcRn-02-IgG4 against human FcRn targets were finally constructed and prepared. The amino acid sequences of the antibodies are shown in Table 1.

In addition, RNA was extracted from human blood samples to establish a yeast display library of human natural scFv antibodies, from which antibodies against human RcRn targets were screened. After further optimization, full-length antibodies FcRn-03-IgG4 and FcRn-04-IgG4 were constructed, and the amino acid sequences of the antibodies were shown in Table 1.

### (2) Inhibition the Recycling Assay of Anti-FCRN Antibody

FcRn has the characteristic of pH dependent binding to IgG antibodies and is a key receptor for maintaining the metabolic level of IgG immunoglobulin in vivo. MDCK cells are model cells for studying FcRn transport IgG. MDCK cells stably transfected with two subunits of human FcRn (hFCGRT and hB2M) can transport human IgG, and anti-FCRN antibodies can block this activity. By measuring the IgG concentration, the inhibitory activity of anti-FCRN antibodies is evaluated. As shown in Table 3, the prepared anti-FcRn antibodies can inhibit the IgG recycling, and is comparable to or even superior to the positive control antibody Rozanolixizumab (UCB, anti FcRn antibody).

TABLE 3
Test results of antibody activity

| Antibodies | IC50(nM) | Antibodies | IC50(nM) |
|---|---|---|---|
| FcRn-01-IgG4 | 11.86 | FcRn-03-IgG4 | 11.46 |
| FcRn-02-IgG4 | 8.358 | FcRn-04-IgG4 | 14.78 |
| Rozanolixizumab | 11.83 | Rozanolixizumab | 12.89 |

### Example 2. Screening of Dual Surfactants in High Concentration Antibody Formulations

In the process of continuous oscillation, the collision probability between antibodies is greatly increased, and in the process of oscillation, the air-solution interface and the bottle wall-solution interface are constantly introduced, which makes the antibodies at the interface easily form polymers or even particles.

This study was conducted to test the effect of surfactants on the stability of antibodies under oscillating condition. The buffer in the prescription was 20 mM acetate, pH was 5.2, and the stabilizer was 60 mg/ml sucrose. The design of the prescription for surfactant screening was shown in Table 4, wherein anti-TSLP antibody took TSLP-01-IGG1 antibody as an example, and anti-FcRn antibody took FcRn-01-IgG4 as an example. The stability of different prescriptions was investigated under oscillating condition (2-8° C., 300 rpm, 2 months), including concentration, pH, osmotic pressure, biological activity (relative to the labeled amount, %), insoluble particles, and aggregation characteristics. Wherein the insoluble particles were detected by Flowcam method, and the polymer characteristics were detected by SEC-HPLC method and NR-CE-SDS method. The test results are shown in Table 5 and 6.

TABLE 4
Design scheme of the prescription for surfactant screening

| Components of | Prescription | | | | | | | | | |
|---|---|---|---|---|---|---|---|---|---|---|
| prescription | 1 | 2 | 3 | 4 | 5 | 6 | 7 | 8 | 9 | 10 |
| TSLP-01-IgG1 | 150 | 160 | 150 | 160 | 150 | | | | | |
| (mg/ml) | | | | | | | | | | |
| FcRn-01-IgG4 | | | | | | 160 | 150 | 160 | 160 | 160 |
| (mg/ml) | | | | | | | | | | |
| PS80 (%, w/v) | 0.02 | 0.03 | 0.15 | | | 0.05 | 0.3 | 0.45 | | |
| PS20 (%, w/v) | | | | | | | | | | |
| P188 (%, w/v) | | | | 0.5 | 1.5 | | | | 0.4 | 1.5 |

TABLE 4
Design scheme of the prescription for surfactant screening(Continued)

| Components of | Prescription | | | | | | | |
|---|---|---|---|---|---|---|---|---|
| prescription | 11 | 12 | 13 | 14 | 15 | 16 | 17 | 18 |
| Anti-TSLP antibody | 150 | 150 | 160 | 150 | | | | |
| (mg/ml) | | | | | | | | |
| Anit-FcRn antibody | | | | | 150 | 150 | 150 | 160 |
| (mg/ml) | | | | | | | | |
| PS80 (mg/ml) | 0.02 | 0.1 | 0.15 | | 0.05 | 0.3 | 0.45 | |
| PS20 (mg/ml) | | | | 0.1 | | | | 0.3 |
| P188 (mg/ml) | 1.5 | 1.0 | 0.5 | 1.0 | 1.5 | 1.0 | 0.4 | 1.0 |

TABLE 5
Visible foreign matter and insoluble particles results
of the prescription for surfactant screening

| Test item | Prescription | 0 | 7 D | 1 M | 2 M |
|---|---|---|---|---|---|
| Visible | 1 | None | Visible foreign | / | / |
| foreign | | | matter | | |
| matter | 2 | None | Visible foreign | / | / |
| | | | matter | | |
| | 3 | None | Visible foreign | / | / |
| | | | matter | | |
| | 4 | None | None | Visible foreign | / |
| | | | | matter | |
| | 5 | None | None | Visible foreign | / |
| | | | | matter | |
| | 6 | None | None | Visible foreign | / |
| | | | | matter | |
| | 7 | None | None | Visible foreign | / |
| | | | | matter | |
| | 8 | None | None | Visible foreign | / |
| | | | | matter | |
| | 9 | None | Visible foreign | / | / |
| | | | matter | | |
| | 10 | None | Visible foreign | / | / |
| | | | matter | | |
| | 11 | None | None | None | None |
| | 12 | None | None | None | None |
| | 13 | None | None | None | None |
| | 14 | None | None | None | None |
| | 15 | None | None | None | None |
| | 16 | None | None | None | None |
| | 17 | None | None | None | None |
| | 18 | None | None | None | None |
| >25 μm | 1 | 0 | 45 | 102 | 203 |
| particles | 2 | 0 | 51 | 98 | 198 |
| (Particles/ | 3 | 0 | 39 | 121 | 186 |
| mL) | 4 | 0 | 31 | 69 | 120 |
| | 5 | 0 | 29 | 59 | 113 |
| | 6 | 0 | 35 | 71 | 121 |
| | 7 | 0 | 40 | 82 | 132 |
| | 8 | 0 | 39 | 88 | 128 |
| | 9 | 0 | 58 | 96 | 193 |
| | 10 | 0 | 57 | 105 | 205 |
| | 11 | 0 | 2 | 6 | 20 |
| | 12 | 0 | 0 | 3 | 14 |
| | 13 | 0 | 2 | 9 | 18 |
| | 14 | 0 | 3 | 10 | 20 |
| | 15 | 0 | 2 | 9 | 23 |
| | 16 | 0 | 0 | 6 | 16 |
| | 17 | 0 | 2 | 12 | 20 |
| | 18 | 0 | 3 | 10 | 18 |
| >10 μm | 1 | 0 | 125 | 258 | 496 |
| particles | 2 | 0 | 119 | 279 | 489 |
| (Particles/ | 3 | 0 | 131 | 264 | 501 |
| mL) | 4 | 0 | 98 | 159 | 313 |
| | 5 | 0 | 89 | 168 | 321 |
| | 6 | 0 | 102 | 148 | 302 |
| | 7 | 0 | 111 | 157 | 308 |
| | 8 | 0 | 106 | 150 | 312 |
| | 9 | 0 | 141 | 301 | 596 |
| | 10 | 0 | 149 | 315 | 600 |
| | 11 | 0 | 57 | 121 | 201 |
| | 12 | 0 | 30 | 99 | 179 |
| | 13 | 0 | 59 | 128 | 190 |
| | 14 | 0 | 65 | 127 | 195 |
| | 15 | 0 | 61 | 119 | 190 |
| | 16 | 0 | 29 | 98 | 185 |
| | 17 | 0 | 52 | 126 | 184 |
| | 18 | 0 | 49 | 121 | 181 |

TABLE 6
Polymer test results of the prescription for surfactant screening

| | | | | | | | | The changes |
|---|---|---|---|---|---|---|---|---|
| | | | | | | | | after 2 M |
| | | | | | | | | compared |
| Test item | | Prescription | 0 D | 3 D | 10 D | 1 M | 2 M | with 0 D |
| SEC | Main | 1 | 99.01 | 90.25 | 83.10 | / | / | >−15% |
| | peak % | 2 | 98.98 | 91.31 | 82.10 | / | / | >−15% |
| | | 3 | 98.57 | 87.91 | 84.28 | / | / | >−15% |
| | | 4 | 99.05 | 98.58 | 98.94 | 98.86 | 94.25 | −4.80 |
| | | 5 | 99.05 | 98.57 | 97.69 | 96.09 | 95.35 | −4.70 |
| | | 6 | 99.08 | 97.59 | 95.32 | 92.19 | 90.18 | −8.90 |
| | | 7 | 99.01 | 98.05 | 96.33 | 92.54 | 90.52 | −8.49 |
| | | 8 | 99.00 | 98.04 | 95.02 | 92.36 | 89.80 | −9.20 |
| | | 9 | 99.03 | 98.78 | 97.99 | 96.39 | 93.75 | −5.28 |
| | | 10 | 99.04 | 97.98 | 96.05 | 95.68 | 93.84 | −5.20 |
| | | 11 | 99.04 | 99.01 | 98.87 | 98.79 | 98.84 | −0.20 |
| | | 12 | 99.01 | 99.00 | 98.89 | 98.86 | 98.83 | −0.18 |
| | | 13 | 99.05 | 99.05 | 99.03 | 98.99 | 98.82 | −0.23 |
| | | 14 | 99.03 | 98.99 | 98.95 | 98.90 | 98.82 | −0.21 |
| | | 15 | 99.01 | 98.98 | 98.91 | 98.85 | 98.79 | −0.22 |
| | | 16 | 99.02 | 98.99 | 98.87 | 98.86 | 98.83 | −0.19 |
| | | 17 | 99.05 | 99.04 | 98.98 | 98.87 | 98.84 | −0.21 |
| | | 18 | 99.03 | 99.01 | 98.87 | 98.86 | 98.83 | −0.20 |
| NR-CE | Main | 1 | 96.5 | 95.1 | 94.2 | 93.9 | 92.3 | −4.2 |
| | peak % | 2 | 96.5 | 95.9 | 95.4 | 93.8 | 92.5 | −4.0 |
| | | 3 | 96.4 | 96.1 | 95.8 | 93.9 | 92.3 | −4.1 |
| | | 4 | 96.2 | 95.8 | 94.7 | 94.1 | 93.2 | −3.0 |
| | | 5 | 96.5 | 96 | 95.4 | 94.9 | 94.0 | −2.5 |
| | | 6 | 96.6 | 96.1 | 95.8 | 94.7 | 93.5 | −3.1 |
| | | 7 | 96.6 | 96.2 | 95.2 | 94.3 | 93.8 | −2.8 |
| | | 8 | 96.3 | 96.1 | 95.6 | 93.9 | 92.3 | −4.0 |
| | | 9 | 96.5 | 96.1 | 95.5 | 93.8 | 92.5 | −4.0 |
| | | 10 | 96.7 | 95.9 | 94.3 | 93.9 | 93.1 | −3.6 |
| | | 11 | 96.5 | 96.6 | 96.5 | 96.3 | 96.2 | −0.3 |
| | | 12 | 96.5 | 96.6 | 96.5 | 96.3 | 96.2 | −0.3 |
| | | 13 | 96.4 | 96.4 | 96.1 | 95.9 | 95.6 | −0.8 |
| | | 14 | 96.5 | 96.1 | 95.9 | 95.8 | 95.6 | −0.9 |
| | | 15 | 96.8 | 96.7 | 96.1 | 95.9 | 95.8 | −1.0 |
| | | 16 | 96.3 | 96.3 | 96.4 | 96.2 | 96.0 | −0.3 |
| | | 17 | 96.5 | 96.1 | 95.8 | 96 | 96 | −0.5 |
| | | 18 | 96.7 | 96.3 | 96.1 | 95.8 | 95.6 | −1.1 |

After 2 months of oscillation, the pH of the antibody formulations in each group was in the range of 5.21-5.25, the osmotic pressure was in the range of 295-300 mOsm, the concentration was in the range of 150 mg/ml-160 mg/ml, and the biological activity of the protein samples (relative to the labeled dose, %) was in the range of 115-130%, and all indexes were basically stable.

The antibody formulations with polysorbate alone or poloxam alone as a surfactant showed visible foreign matter at the seventh day or the first month. Flowcam test results showed that at the second month, compared with the zero month, the number of >25 μm particles increased by up to 113-205, and the number of >10 μm particles increased by up to 302-600; the main peak by SEC of prescription 1-3 groups was reduced to less than 85% at the tenth day, indicating that the prescription formulations produced a large number of polymers; at the second month, the main peak by SEC of prescription 4-10 groups decreased by 4.70-9.20% compared with the zero month; and the main peak by NR-CE decreased by 2.5-4.2% at the second month compared with the zero month.

In contrast, no visible foreign matter was found in the antibody formulations with polysorbate and Poloxam after 2 months of oscillation. Flowcam detection showed that at the second month, compared with the zero month, the number of >25 μm particles was only 14-23, and the number of >10 μm particles was only 179-201; at the second month, the main peak by SEC-HPLC decreased by 0.18-0.23% compared with the zero month; and at the second month, the main peak by NR-CE decreased by 0.18-0.23% compared with the zero month.

The above results indicate that for high concentration antibody formulations, under the condition of oscillation, the single surfactant (polysorbide or Poloxam) can not effectively prevent the formation of polymers, and thus can not effectively maintain the stability of antibodies. However, the combination of polysorbate and poloxam in a high-concentration antibody formulation can maintain the stability of the antibody more effectively than a single surfactant.

### Example 3. Prescription Screening of High-Concentration Antibody Formulations

In this study, the prescription of high-concentration antibody formulation was further screened under the condition of oscillation. The combination of surfactants in the formulation was 0.3 mg/ml polysorbate 80 and 1.0 mg/ml Poloxam 188, and the design of the screening prescription was shown in Table 7. The stability of different prescriptions was investigated under oscillating condition (2-8° C., 300 rpm, 2 months), including concentration, pH, osmotic pressure, biological activity (relative to the labeled amount, %), insoluble particles, and aggregation characteristics. The insoluble particles were detected by Flowcam method, and the polymer characteristics were detected by SEC-HPLC method and NR-CE-SDS method. The test results are shown in Table 8 and 9.

TABLE 7
Design scheme of the prescription screening

| | Component of | Prescription | | | | | | | | | | | | | | |
|---|---|---|---|---|---|---|---|---|---|---|---|---|---|---|---|---|
| Use | presciption | 1 | 2 | 3 | 4 | 5 | 6 | 7 | 8 | 9 | 10 | 11 | 12 | 13 | 14 | 15 |
| Antibody | TSLP-01- | 150 | 150 | 135 | 150 | 165 | 150 | 150 | 160 | 150 | 150 | | | | | |
| | IgG1 | | | | | | | | | | | | | | | |
| | mg/ml | | | | | | | | | | | | | | | |
| | FcRn-01- | | | | | | | | | | | 135 | 150 | 165 | 150 | 160 |
| | IgG4 | | | | | | | | | | | | | | | |
| | mg/ml | | | | | | | | | | | | | | | |
| Stabilizer | Sucrose | | | 50 | 60 | 70 | 80 | | 60 | 60 | 60 | 50 | 60 | 70 | | 60 |
| | mg/ml | | | | | | | | | | | | | | | |
| | Trehalose | | 60 | | | | | | | | | | | | | |
| | mg/ml | | | | | | | | | | | | | | | |
| | NaCl | 130 | | | | | | | | | | | | | 130 | |
| | mM | | | | | | | | | | | | | | | |
| | Arginine | | | | | | | 150 | | | | | | | | |
| | mM | | | | | | | | | | | | | | | |
| Buffer | Acetate | 20 | | 10 | 20 | 30 | 30 | 20 | | | | 10 | 20 | 30 | 20 | |
| | mM | | | | | | | | | | | | | | | |
| | Histidine | | | | | | | | | | 20 | | | | | |
| | hydro- | | | | | | | | | | | | | | | |
| | chloridemM | | | | | | | | | | | | | | | |
| | Citrate | | 20 | | | | | | 20 | 20 | | | | | | 20 |
| | mM | | | | | | | | | | | | | | | |
| Polysorbate | PS80 | 0.1 | 0.1 | 0.1 | 0.1 | 0.1 | 0.1 | 0.1 | 0.1 | 0.1 | 0.1 | 0.3 | 0.3 | 0.3 | 0.3 | 0.3 |
| Poloxamer | P188 | 1.0 | 1.0 | 1.0 | 1.0 | 1.0 | 1.0 | 1.0 | 1.0 | 1.0 | 1.0 | 1.0 | 1.0 | 1.0 | 1.0 | 1.0 |
| Antioxidant | Methionine | | | | | | | | 20 | | | | | | | 20 |
| | mM | | | | | | | | | | | | | | | |
| pH Value | pH Value | 5.4 | 5.0 | 5.4 | 5.2 | 5.0 | 5.4 | 5.2 | 5.0 | 4.8 | 5.5 | 5.4 | 5.2 | 5.0 | 5.4 | 5.0 |

TABLE 8
Visible foreign matter and insoluble
particles results of the prescription

| Test item | Prescription | 0 | 7 D | 1 M | 2 M |
|---|---|---|---|---|---|
| Visible | 1 | None | None | None | None |
| foreign | 2 | None | None | None | None |
| matter | 3 | None | None | None | None |
| | 4 | None | None | None | None |
| | 5 | None | None | None | None |
| | 6 | None | None | None | None |
| | 7 | None | None | None | None |
| | 8 | None | None | None | None |
| | 9 | None | None | None | None |
| | 10 | None | None | None | None |
| | 11 | None | None | None | None |
| | 12 | None | None | None | None |
| | 13 | None | None | None | None |
| | 14 | None | None | None | None |
| | 15 | None | None | None | None |
| >25μ | 1 | 0 | 5 | 19 | 50 |
| particles | 2 | 0 | 8 | 20 | 55 |
| (Particles/mL) | 3 | 0 | 1 | 5 | 17 |
| | 4 | 0 | 0 | 3 | 14 |
| | 5 | 0 | 1 | 6 | 15 |
| | 6 | 0 | 23 | 32 | 78 |
| | 7 | 0 | 10 | 20 | 49 |
| | 8 | 0 | 9 | 23 | 61 |
| | 9 | 0 | 20 | 41 | 81 |
| | 10 | 0 | 25 | 39 | 85 |
| | 11 | 0 | 2 | 7 | 19 |
| | 12 | 0 | 0 | 6 | 16 |
| | 13 | 0 | 1 | 7 | 20 |
| | 14 | 0 | 3 | 29 | 62 |
| | 15 | 0 | 9 | 31 | 63 |
| >10 μm | 1 | 0 | 60 | 132 | 255 |
| particles | 2 | 0 | 59 | 129 | 264 |
| (Particles/mL) | 3 | 0 | 32 | 101 | 191 |
| | 4 | 0 | 30 | 99 | 179 |
| | 5 | 0 | 36 | 112 | 180 |
| | 6 | 0 | 78 | 156 | 256 |
| | 7 | 0 | 62 | 134 | 226 |
| | 8 | 0 | 64 | 129 | 249 |
| | 9 | 0 | 80 | 160 | 251 |
| | 10 | 0 | 82 | 158 | 249 |
| | 11 | 0 | 34 | 110 | 193 |
| | 12 | 0 | 29 | 98 | 185 |
| | 13 | 0 | 32 | 117 | 184 |
| | 14 | 0 | 58 | 140 | 276 |
| | 15 | 0 | 50 | 147 | 215 |

TABLE 9
Polymer test results of the prescription

| | | | | | | | | Changes at |
|---|---|---|---|---|---|---|---|---|
| | | | | | | | | 2 M |
| | | | | | | | | compared to |
| Test item | | Prescription | 0 | 3 D | 10 D | 1 M | 2 M | 0 D |
| SEC | Main | 1 | 99.01 | 98.86 | 98.01 | 97.75 | 96.91 | −2.10 |
| | peak % | 2 | 99.02 | 98.86 | 97.98 | 97.01 | 96.72 | −2.30 |
| | | 3 | 99.05 | 99.01 | 98.89 | 98.85 | 98.85 | −0.20 |
| | | 4 | 99.01 | 99.00 | 98.89 | 98.86 | 98.83 | −0.18 |
| | | 5 | 99.03 | 99.01 | 98.99 | 98.85 | 98.80 | −0.23 |
| | | 6 | 99.89 | 97.25 | 96.98 | 96.6 | 95.89 | −4.00 |
| | | 7 | 98.99 | 98.82 | 97.63 | 95.98 | 95.49 | −3.50 |
| | | 8 | 99.00 | 98.79 | 98.03 | 97.54 | 96.00 | −3.00 |
| | | 9 | 99.01 | 96.59 | 96.35 | 95.70 | 95.11 | −3.90 |
| | | 10 | 99.05 | 98.23 | 98.15 | 97.12 | 95.25 | −3.80 |
| | | 11 | 99.04 | 99.01 | 98.95 | 98.89 | 98.83 | −0.21 |
| | | 12 | 99.02 | 98.99 | 98.87 | 98.86 | 98.83 | −0.19 |
| | | 13 | 99.00 | 98.87 | 98.86 | 98.81 | 98.78 | −0.22 |
| | | 14 | 99.05 | 98.86 | 97.56 | 97.05 | 96.45 | −2.6 |
| | | 15 | 99.05 | 98.74 | 97.58 | 95.69 | 94.55 | −4.5 |
| NR-CE | Main | 1 | 96.5 | 96.4 | 96.0 | 95.8 | 95.2 | −1.3 |
| | peak % | 2 | 96.7 | 96.2 | 95.7 | 95.3 | 95.2 | −1.5 |
| | | 3 | 96.4 | 96.2 | 96.1 | 95.8 | 95.6 | −0.8 |
| | | 4 | 96.5 | 96.6 | 96.5 | 96.3 | 96.2 | −0.3 |
| | | 5 | 96.8 | 96.8 | 96.7 | 96.5 | 96.4 | −0.4 |
| | | 6 | 96.3 | 95.6 | 95.1 | 94.5 | 94.3 | −2.0 |
| | | 7 | 96.1 | 96.1 | 95.4 | 94.9 | 94.5 | −1.6 |
| | | 8 | 96.2 | 96.1 | 95.3 | 94.8 | 94.7 | −1.5 |
| | | 9 | 96.3 | 95.9 | 95.1 | 95 | 94.4 | −1.9 |
| | | 10 | 96.5 | 96.1 | 95.2 | 94.8 | 94.7 | −1.8 |
| | | 11 | 96.4 | 96.2 | 95.9 | 95.6 | 95.5 | −0.9 |
| | | 12 | 96.3 | 96.3 | 96.4 | 96.2 | 96.0 | −0.3 |
| | | 13 | 96.2 | 96.2 | 95.9 | 95.6 | 95.3 | −0.9 |
| | | 14 | 96.5 | 96.1 | 95.7 | 95.1 | 94.7 | −1.8 |
| | | 15 | 96.2 | 95.8 | 95.2 | 94.9 | 94.6 | −1.6 |

After 2 months of oscillation, there was no visible foreign matter in antibody formulations in each group, and the pH, osmotic pressure, protein concentration, and biological activity of protein samples (relative to labeling dose) were basically stable in each group.

Flowcam test results showed that at the second month, compared with the zero day, the number of >25 μm particles increased by 14-85 and the number of >10 μm particles increased by 179-276 in each group, which were superior to the single surfactant prescription only. In contrast, the number of >25 μm particles increased by only 14-20, and the number of >10 μm particles increased by only 179-193, showing a better stability effect.

The results of SEC-HPLC showed that at the second month, compared with the zero day, the main peak of antibody formulation in each group decreased by 0.18%- 4.5%, which was better than that of single surfactant only. In contrast, the main peak of antibody formulations in prescription groups 3-5 and 11-13 was only reduced by 0.18-0.23%, showing a better stability effect. The results of NR-CE test also showed the same conclusion.

In summary, using the combination of polysorbate and Poloxam, all antibody formulations in each group maintained good stability, especially prescriptions 3-5 and 11-13, which had better stability effect.

### Example 4. Confirmation of High-Concentration Antibody Preparation

According to the above examples, select one of the prescriptions and take it as an example to prepare high-concentration liquid formulations of antibodies with different sequences, and test the stability of these antibody formulations under repeated freeze-thaw, oscillation, acceleration, and long-term conditions. Among them, the difference between FcRn-01-IgG4 (S228P) and FcRn-01-IgG4 is only that the heavy chain constant region of the former is SEQ ID NO: 13, while the latter is SEQ ID NO: 14.

TABLE 10
Confirmation of high concentration antibody formulations

| | | Concentration | | | | | |
|---|---|---|---|---|---|---|---|
| | | of antibody | | | Stabilizer | PS80 | P188 |
| | Antibody | (mg/ml) | Buffer | pH | (mg/ml) | (mg/ml) | (mg/ml) |
| 1 | TSLP-01-IgG1 | 150 | 20 Mm | 5.2 | 60 mg/ml | 0.1 | 1.0 |
| | | | HAC | | Sucrose | | |
| 2 | TSLP-02-IgG1 | 150 | 20 Mm | 5.2 | 60 mg/ml | 0.1 | 1.0 |
| | | | HAC | | Sucrose | | |
| 3 | FcRn-01-IgG4 | 150 | 20 Mm | 5.2 | 60 mg/ml | 0.3 | 1.0 |
| | (S228P) | | HAC | | Sucrose | | |
| 4 | FcRn-02-IgG4 | 150 | 20 Mm | 5.2 | 60 mg/ml | 0.3 | 1.0 |
| | | | HAC | | Sucrose | | |
| 5 | FcRn-03-IgG4 | 150 | 20 Mm | 5.2 | 60 mg/ml | 0.3 | 1.0 |
| | | | HAC | | Sucrose | | |
| 6 | FcRn-04-IgG4 | 150 | 20 Mm | 5.2 | 60 mg/ml | 0.3 | 1.0 |
| | | | HAC | | Sucrose | | |

### (1) Repeated Freeze-Thaw Stability Test

The stability of the antibody formulation was tested under freeze-thaw conditions (−80° C. freezing/room temperature melting, 5 cycles). Table 11 shows the test results of each antibody formulation: no visible foreign matter was found within 5 times of freeze-thaw. Compared with the zero time of freeze-thaw, the concentration, pH, osmotic pressure, biological activity and insoluble particles of antibody preparation basically did not change; the main peaks changed little by SEC, all within 0.07%; the variation range of the main peak by NR-CE were within 0.5%. The above results indicated the excellent stability of different antibody formulations of the present application under freeze-thaw conditions.

TABLE 11
Freeze-thaw test results of antibody formulations

| | | | | | | | Changes at |
|---|---|---|---|---|---|---|---|
| | | | | | | | freeze-thaw |
| | | | | | | | 5 times |
| | | | Freeze- | Freeze- | Freeze- | Freeze- | compared to |
| | | | thaw 0 | thaw 1 | thaw 3 | thaw 5 | freeze-thaw |
| Test item | | Prescription | time | time | times | times | 0 time |
| SEC | Main | 1 | 99.05 | 99.01 | 98.89 | 99.00 | −0.05 |
| | peak % | 2 | 99.03 | 99.01 | 99.00 | 99.00 | −0.03 |
| | | 3 | 99.03 | 99.03 | 99.01 | 98.98 | −0.05 |
| | | 4 | 99.05 | 99.10 | 98.99 | 98.98 | −0.07 |
| | | 5 | 99.07 | 99.02 | 99.02 | 99.01 | −0.06 |
| | | 6 | 99.02 | 98.99 | 98.97 | 98.97 | −0.05 |
| NR-CE | Main | 1 | 96.5 | 96.4 | 96.3 | 96.3 | −0.2 |
| | peak % | 2 | 96.5 | 96.5 | 96.4 | 96.1 | −0.4 |
| | | 3 | 96.6 | 96.3 | 96.3 | 96.1 | −0.5 |
| | | 4 | 96.4 | 96.4 | 96.2 | 96.2 | −0.2 |
| | | 5 | 96.2 | 96.3 | 96.4 | 95.9 | −0.3 |
| | | 6 | 96.5 | 96.5 | 96.4 | 96.2 | −0.3 |

### (2) Oscillation Stability Test:

The stability of the antibody formulation was tested under oscillatory conditions (2-8° C., 300 rpm, standing). Table 12 shows the test results of each antibody formulation: Each formulation also showed excellent stability under the condition of oscillation: no visible foreign matter was found within 2 months of oscillation; compared with the zero day, the concentration, pH, osmotic pressure, biological activity and insoluble particles of antibody formulation basically did not change; the main peak changed very little by SEC, all within 0.30%; the variation range of the main peak by NR-CE were within 0.7%. The above results indicate the excellent stability of the antibody formulation under oscillatory conditions.

TABLE 12
Results of oscillation tests for antibody formulations

| | | | | | | | | Changes at 2 M |
|---|---|---|---|---|---|---|---|---|
| | | | | | | | | compared to |
| | | | | | | | | 0 D under |
| | | | | | | | | oscillation |
| Test item | | Prescription | 0 D | 10 D | 1 M | 1.5 M | 2 M | conditions |
| SEC | Main | 1 | 99.05 | 99.01 | 98.98 | 98.88 | 98.84 | −0.21 |
| | peak % | 2 | 99.07 | 99.07 | 99.05 | 98.99 | 98.78 | −0.29 |
| | | 3 | 99.03 | 99.01 | 98.89 | 98.88 | 98.88 | −0.15 |
| | | 4 | 99.05 | 99.04 | 99.01 | 98.78 | 98.75 | −0.30 |
| | | 5 | 99.06 | 99.05 | 98.99 | 98.87 | 98.81 | −0.25 |
| | | 6 | 99.07 | 99.06 | 99.01 | 98.96 | 98.81 | −0.26 |
| NR-CE | Main | 1 | 96.5 | 96.5 | 96.4 | 96.4 | 96.1 | −0.4 |
| | peak % | 2 | 96.3 | 96.1 | 95.8 | 95.6 | 95.6 | −0.7 |
| | | 3 | 96.4 | 96.5 | 96.4 | 96.1 | 96.1 | −0.3 |
| | | 4 | 96.2 | 96.1 | 95.7 | 95.6 | 95.7 | −0.5 |
| | | 5 | 96.7 | 96.7 | 96.3 | 96.2 | 96.1 | −0.6 |
| | | 6 | 96.2 | 96.1 | 95.8 | 95.7 | 95.5 | −0.7 |

### (3) Acceleration Stability:

The stability of the antibody formulation was tested under accelerated conditions (25° C., 2 months). Table 13 shows the test results of each antibody formulation: within 2 months of acceleration, no visible foreign matter was found, compared with the zero day, the concentration, pH, osmotic pressure, biological activity and insoluble particles of antibody formulation basically did not change; the main peaks by SEC were less than 1.09%; the variation range of the main peak by NR-CE were less than 1.30%. The above results indicate that the formulation of the antibody of the present application can maintain excellent stability under accelerated conditions.

TABLE 13
Results of accelerated tests of antibody formulations

| | | | | | | | Changes |
|---|---|---|---|---|---|---|---|
| | | | | | | | at 2 M |
| | | Prescrip- | | | | | compared |
| Test item | | tion | 0 D | 1 M | 1.5 M | 2 M | to 0 D |
| SEC | Main | 1 | 99.04 | 99.01 | 98.85 | 98.15 | −0.89 |
| | peak | 2 | 99.05 | 99.03 | 98.56 | 97.96 | −1.09 |
| | % | 3 | 99.06 | 99.00 | 98.52 | 98.37 | −0.69 |
| | | 4 | 99.04 | 98.98 | 98.26 | 98.04 | −1.00 |
| | | 5 | 99.07 | 99.04 | 98.56 | 98.05 | −1.02 |
| | | 6 | 99.02 | 98.67 | 98.12 | 97.99 | −1.03 |
| NR- | Main | 1 | 96.5 | 96.2 | 95.6 | 95.6 | −0.9 |
| CE | peak | 2 | 96.5 | 96.3 | 95.4 | 95.2 | −1.3 |
| | % | 3 | 96.3 | 95.7 | 95.6 | 95.5 | −0.8 |
| | | 4 | 96.4 | 96.1 | 95.7 | 95.2 | −1.2 |
| | | 5 | 96.2 | 95.5 | 95.2 | 95.2 | −1.0 |
| | | 6 | 96.5 | 96.2 | 95.8 | 95.4 | −1.1 |

### (4) Long-Term Stability:

The stability of the antibody formulation was tested under long-term conditions (2-8° C., standing, 6 months). Table 14 shows the test results of each antibody formulation: after 6 months under long-term conditions, no visible foreign matter was found, and compared with the zero day, the concentration, pH, osmotic pressure, biological activity and insoluble particles of antibody preparation basically did not change. The main peaks by SEC changed little, all within 0.18%; the variation range of the main peak by NR-CE were within 0.4%. The above results indicate that the formulation of the antibody of the present application can maintain excellent stability under long-term conditions.

TABLE 14
Long-term test results of antibody formulations

| | | | | | | | | Changes at 6 M |
|---|---|---|---|---|---|---|---|---|
| Test item | | Prescription | 0 D | 1 M | 2 M | 3 M | 6 M | compared to 0 D |
| SEC | Main | 1 | 99.05 | 99.03 | 98.96 | 98.97 | 98.96 | −0.09 |
| | peak % | 2 | 99.04 | 99.02 | 99.01 | 98.89 | 98.86 | −0.18 |
| | | 3 | 99.03 | 99.03 | 99.01 | 98.88 | 98.94 | −0.09 |
| | | 4 | 99.01 | 98.99 | 98.87 | 98.87 | 98.85 | −0.16 |
| | | 5 | 99.03 | 99.01 | 98.99 | 98.89 | 98.95 | −0.08 |
| | | 6 | 99.05 | 99.04 | 98.97 | 98.91 | 98.90 | −0.15 |
| NR-CE | Main | 1 | 96.4 | 96.4 | 96.1 | 95.9 | 96.2 | −0.2 |
| | peak % | 2 | 96.6 | 96.4 | 96.2 | 96.3 | 96.3 | −0.3 |
| | | 3 | 96.5 | 96.4 | 96.3 | 96.3 | 96.3 | −0.2 |
| | | 4 | 96.4 | 96.3 | 96.2 | 96.2 | 96.1 | −0.3 |
| | | 5 | 96.3 | 96.3 | 96.2 | 95.9 | 95.9 | −0.4 |
| | | 6 | 96.4 | 96.2 | 96.1 | 96.2 | 96.1 | −0.3 |

In summary, the high-concentration antibody formulation of the present application has strong stability under freeze-thaw, oscillation, acceleration and long-term conditions, is suitable for various sequences of antibodies, and can maintain good stability during preparation, transportation and storage, ensuring the safety of clinical drug use and quality control.

1. A high-concentration antibody formulation, wherein the antibody formulation comprises a monoclonal antibody and surfactants, wherein the surfactants are polysorbate and poloxamer, and the concentration of the monoclonal antibody is from 100 mg/ml to 200 mg/ml, optionally from 135 mg/ml to 165 mg/ml.

2. The antibody formulation of claim 1, wherein the polysorbate is polysorbate 20 and/or polysorbate 80; optionally, the poloxamer is poloxamer 188.

3. The antibody formulation of claim 1, wherein the concentration of the polysorbate is from 0.02 mg/ml to 0.45 mg/ml, optionally from 0.08 mg/ml to 0.4 mg/ml, more optionally from 0.1 mg/ml to 0.3 mg/ml.

4. The antibody formulation of claim 1, wherein the concentration of the poloxamer is from 0.4 to 1.5 mg/ml, optionally from 0.8 mg/ml to 1.2 mg/ml, more preferably 1.0 mg/ml.

5. The antibody formulation of claim 1, wherein the antibody formulation further comprises a stabilizer, optionally, the stabilizer is any one or more of sucrose, trehalose, maltose, sorbitol, mannitol, sodium chloride, arginine, glycine, proline and lysine; more optionally, the stabilizer is any one or more of sucrose, trehalose, sodium chloride or arginine.

6. The formulation of claim 5, wherein the stabilizer is sodium chloride, arginine, glycine, proline or lysine at a concentration of from 100 mM to 180 mM, optionally from 130 mM to 150 mM; or sucrose, trehalose, maltose, sorbitol and/or mannitol at a concentration of from 40 mg/ml to 80 mg/ml, optionally from 50 mg/ml to 80 mg/ml, and further preferably from 50 mg/ml to 70 mg/ml.

7. The antibody formulation of claim 1, wherein the antibody formulation further comprises a buffer, optionally the buffer is citrate buffer, acetate buffer and/or histamine buffer.

8. The antibody formulation of claim 7, wherein the concentration of the buffer is from 3 mM to 50 mM; optionally the concentration of the buffer is from 10 mM to 30 mM.

9. The antibody formulation of claim 1, wherein the pH of the formulation is from 4.8 to 5.5, optionally from 5.0 to 5.4.

10. The antibody formulation of claim 1, wherein the concentration of the antibody is from 100 mg/ml to 200 mg/ml; the stabilizer is sucrose and/or trehalose of from 40 mg/ml to 80 mg/ml, or the stabilizer is sodium chloride or arginine of from 100 mM to 180 mM; the buffer is acetate buffer, citrate buffer and/or histidine buffer of from 3 mM to 50 mM; the pH value of the formulation is 4.8-5.5; the surfactants are polysorbate of from 0.02 mg/ml to 0.45 mg/ml and poloxamer of from 0.4 mg/ml to 1.5 mg/ml.

11. The antibody formulation of claim 1, wherein the concentration of the antibody is from 135 mg/ml to 165 mg/ml; the stabilizer is sucrose and/or trehalose of from 50 mg/ml to 80 mg/ml, or the stabilizer is sodium chloride or arginine of from 130 mM to 150 mM; the buffer is acetate buffer, citrate buffer and/or histidine buffer of from 10 mM to 30 mM; the pH value of the formulation is from 4.8 to 5.5; the surfactants are polysorbate of from 0.02 mg/ml to 0.45 mg/ml and poloxamer of from 0.4 mg/ml to 1.5 mg/ml.

12. The antibody formulation of claim 1, wherein the concentration of the antibody is from 135 mg/ml to 165 mg/ml; the stabilizer is sucrose and/or trehalose of from 50 mg/ml to 70 mg/ml, or the stabilizer is sodium chloride or arginine of from 130 mM to 150 mM; the buffer is acetate buffer, citrate buffer and/or histidine buffer of from 10 mM to 30 mM; the pH value of the formulation is from 5.0 to 5.4; the surfactants are polysorbate of from 0.02 mg/ml to 0.45 mg/ml and poloxamer of from 0.4 mg/ml to 1.5 mg/ml.

13. The antibody formulation of claim 1, wherein the formulation is any one of the following antibody formulations:
(1) the concentration of the antibody is 135 mg/ml, 140 mg/ml, 145 mg/ml, 150 mg/ml, 155 mg/ml, 160 mg/ml or 165 mg/ml; the stabilizer is sucrose and/or trehalose of from 50 mg/ml to 70 mg/ml, or the stabilizer is sodium chloride or arginine of from 130 mM to 150 mM; the buffer is acetate buffer, citrate buffer and/or histidine buffer of from 10 mM to 30 mM; the pH value of the formulation is from 5.0 to 5.4; the surfactants are polysorbate of from 0.02 mg/ml to 0.45 mg/ml and poloxamer of from 0.4 mg/ml to 1.5 mg/ml;
(2) the concentration of the antibody is from 135 mg/ml to 165 mg/ml; the stabilizer is sucrose and/or trehalose of 50 mg/ml, 60 mg/ml or 70 mg/ml, or the stabilizer is sodium chloride or arginine of 130 mM, 140 mM or 150 mM; the buffer is acetate buffer, citrate buffer and/or histidine buffer of from 10 mM to 30 mM; the pH value of the formulation is from 5.0 to 5.4; the surfactants are polysorbate of from 0.02 mg/ml to 0.45 mg/ml and poloxamer of from 0.4 mg/ml to 1.5 mg/ml;
(3) the concentration of the antibody is from 135 mg/ml to 165 mg/ml; the stabilizer is sucrose and/or trehalose of from 50 mg/ml to 70 mg/ml, or the stabilizer is sodium chloride or arginine of from 130 mM to 150 mM; the buffer is acetate buffer, citrate buffer and/or histidine buffer of 10 mM, 20 mM or 30 mM; the pH value of the formulation is from 5.0 to 5.4; the surfactants are polysorbate of from 0.02 mg/ml to 0.45 mg/ml and poloxamer of from 0.4 mg/ml to 1.5 mg/ml;
(4) the concentration of the antibody is from 135 mg/ml to 165 mg/ml; the stabilizer is sucrose and/or trehalose of from 50 mg/ml to 70 mg/ml, or the stabilizer is sodium chloride or arginine of from 130 mM to 150 mM; the buffer is acetate buffer, citrate buffer and/or histidine buffer of from 10 mM to 30 mM; the pH value of the formulation is 5.0, 5.1, 5.2, 5.3 or 5.4; the surfactants are polysorbate of from 0.02 mg/ml to 0.45 mg/ml and poloxamer of from 0.4 mg/ml to 1.5 mg/ml;
(5) the concentration of the antibody is from 135 mg/ml to 165 mg/ml; the stabilizer is sucrose and/or trehalose of from 50 mg/ml to 70 mg/ml, or the stabilizer is sodium chloride or arginine of from 130 mM to 150 mM; the buffer is acetate buffer, citrate buffer and/or histidine buffer of from 10 mM to 30 mM; the pH value of the formulation is from 5.0 to 5.4; the surfactants are polysorbate of 0.02 mg/ml, 0.03 mg/ml, 0.04 mg/ml, 0.05 mg/ml, 0.06 mg/ml, 0.08 mg/ml, 0.10 mg/ml, 0.12 mg/ml, 0.15 mg/ml, 0.20 mg/ml, 0.30 mg/ml, 0.40 mg/ml or 0.45 mg/ml and poloxamer of from 0.4 mg/ml to 1.5 mg/ml;
(6) the concentration of the antibody is from 135 mg/ml to 165 mg/ml; the stabilizer is sucrose and/or trehalose of from 50 mg/ml to 70 mg/ml, or the stabilizer is sodium chloride or arginine of from 130 mM to 150 mM; the buffer is acetate buffer, citrate buffer and/or histidine buffer of from 10 mM to 30 mM; the pH value of the formulation is from 5.0 to 5.4; the surfactants are polysorbate of from 0.02 mg/ml to 0.45 mg/ml and poloxamer of 0.4 mg/ml, 0.5 mg/ml, 0.6 mg/ml, 0.8 mg/ml, 1.0 mg/ml or 1.2 mg/ml 1.5 mg/ml; and
the polysorbate is polysorbate 80 or polysorbate 20.

14. The antibody formulation of claim 1, wherein the formulation is any one of the following antibody formulations:
(1) the concentration of the antibody is 150 mg/ml; the stabilizer is sodium chloride of 130 mM; the buffer is acetate buffer of 20 mM; the pH value of the formulation is 5.4; the surfactants are polysorbate of 0.1 mg/ml or 0.3 mg/ml and poloxamer of 1.0 mg/ml;
(2) the concentration of the antibody is 150 mg/ml; the stabilizer is sucrose of 60 mg/ml; the buffer is citrate buffer of 20 mM; the pH value of the formulation is 5.0; the surfactants are polysorbate of 0.1 mg/ml or 0.3 mg/ml and poloxamer of 1.0 mg/ml;
(3) the concentration of the antibody is 135 mg/ml; the stabilizer is sucrose of 50 mg/ml; the buffer is acetate buffer of 10 mM; the pH value of the formulation is 5.4; the surfactants are polysorbate of 0.1 mg/ml or 0.3 mg/ml and poloxamer of 1.0 mg/ml;
(4) the concentration of the antibody is 150 mg/ml; the stabilizer is sucrose of 60 mg/ml; the buffer is acetate buffer of 20 mM; the pH value of the formulation is 5.2; the surfactants are polysorbate of 0.1 mg/ml or 0.3 mg/ml and poloxamer of 1.0 mg/ml;
(5) the concentration of the antibody is 165 mg/ml; the stabilizer is the sucrose of 70 mg/ml; the buffer is the acetate buffer of 30 mM; the pH value of the formulation is 5.0; the surfactants are polysorbate of 0.1 mg/ml or 0.3 mg/ml and poloxamer of 1.0 mg/ml;
(6) the concentration of the antibody is 150 mg/ml; the stabilizer is the sucrose of 80 mg/ml; the buffer is the acetate buffer of 30 mM; the pH value of the formulation is 5.4; the surfactants are polysorbate of 0.1 mg/ml or 0.3 mg/ml and poloxamer of 1.0 mg/ml;
(7) the concentration of the antibody is 150 mg/ml; the stabilizer is arginine of 150 mM; the buffer is acetate buffer of 20 mM; the pH value of the formulation is 5.2; the surfactants are polysorbate of 0.1 mg/ml or 0.3 mg/ml and poloxamer of 1.0 mg/ml;
(8) the concentration of the antibody is 160 mg/ml; the stabilizer is sucrose of 60 mg/ml; the buffer is citrate buffer of 20 mM; the pH value of the formulation is 5.0; the surfactants are polysorbate of 0.1 mg/ml or 0.3 mg/ml and poloxamer of 1.0 mg/ml;
(9) the concentration of the antibody is 150 mg/ml; the stabilizer is the sucrose of 60 mg/ml; the buffer is the citrate buffer of 20 mM; the pH value of the formulation is 4.8; the surfactants are polysorbate of 0.1 mg/ml or 0.3 mg/ml and poloxamer of 1.0 mg/ml;
(10) the concentration of the antibody is 150 mg/ml; the stabilizer is the sucrose of 60 mg/ml; the buffer is the histamine salt buffer of 20 mM; the pH value of the formulation is 5.5; the surfactants are polysorbate of 0.1 mg/ml or 0.3 mg/ml and poloxamer of 1.0 mg/ml;
(11) the concentration of the antibody is 150 mg/ml; the stabilizer is sucrose of 60 mg/ml; the buffer is histamine salt buffer of 20 mM; the pH value of the formulation is 5.2; the surfactants are polysorbate of 0.02 mg/ml or 0.05 mg/ml and poloxamer of 1.5 mg/ml;
(12) the concentration of the antibody is 150 mg/ml; the stabilizer is the sucrose of 60 mg/ml; the buffer is the histidine salt buffer of 20 mM; the pH value of the formulation is 5.2; the surfactants are polysorbate of 0.15 mg/ml and poloxamer of 0.5 mg/ml;
(13) the concentration of the antibody is 150 mg/ml; the stabilizer is the sucrose of 60 mg/ml; the buffer is the histidine salt buffer of 20 mM; the pH value of the formulation is 5.2; the surfactants are polysorbate of 0.45 mg/ml and poloxamer of 0.4 mg/ml;
the polysorbate is polysorbate 80 or polysorbate 20.

15. The antibody formulation of claim 1, wherein the antibody formulation further comprises an antioxidant and/or a preservative.

16. The antibody formulation of claim 1, wherein the antibody formulation is a liquid formulation or a powder injection for injection.

17. The antibody formulation of claim 1, wherein the antibody is an anti-TSLP antibody or an anti-FcRn antibody.

18. The use of the antibody formulation of claim 1 in the preparation of medicament for the treatment of diseases; optionally, the diseases include inflammatory or autoimmune diseases.

19. The antibody formulation of claim 17, wherein the anti-TSLP antibody comprises:
a VH comprising the amino acid sequence of SEQ ID NO: 1; and a VL comprising the amino acid sequence of SEQ ID NO:2; or
a VH comprising the amino acid sequence of SEQ ID NO: 3; and a VL comprising the amino acid sequence of SEQ ID NO:4; and
anti-FcRn antibody comprises:
a VH comprising the amino acid sequence of SEQ ID NO: 5; and a VL comprising the amino acid sequence of SEQ ID NO:6; or
a VH comprising the amino acid sequence of SEQ ID NO: 7; and a VL comprising the amino acid sequence of SEQ ID NO:8; or
a VH comprising the amino acid sequence of SEQ ID NO: 9; and a VL comprising the amino acid sequence of SEQ ID NO:10; or
a VH comprising the amino acid sequence of SEQ ID NO: 11; and a VL comprising the amino acid sequence of SEQ ID NO:10.

20. The antibody formulation of claim 19, wherein the anti-TSLP antibody comprises:
a heavy chain constant region comprising the amino acid sequence of SEQ ID NO: 12, and a light chain constant region comprising the amino acid sequence of SEQ ID NO: 15;
and anti-FcRn antibody comprises:
a heavy chain constant region comprising the amino acid sequence of SEQ ID NO: 13 or 14, and a light chain constant region comprising the amino acid sequence of SEQ ID NO: 15.
